(19) United States
(12) Patent Application Publication
(10) Pub. No.: US 2023/0201454 A1
(43) Pub. Date: Jun. 29, 2023

(54) IMPLANTABLE INTRATHECAL DRUG DELIVERY SYSTEM FOR CHRONIC PAIN CONTROL

(71) Applicants: Board of Regents, The University of Texas System, Austin, TX (US); Bexar Biomedical, San Antonio, TX (US)

(72) Inventors: RALPH JOHNSTON, San Antonio, TX (US); PAUL FENTON, San Antonio, TX (US); DOUG FRANTZ, San Antonio, TX (US); DANIEL PORTILLO, San Antonio, TX (US); SAKETH RAM, San Antonio, TX (US); ROBERT LYLE HOOD, San Antonio, TX (US)

(21) Appl. No.: 17/928,248

(22) PCT Filed: May 29, 2021
(86) PCT No.: PCT/US2021/035047

§ 371 Date: Nov. 28, 2022

Related U.S. Application Data
(60) Provisional application No. 63/032,359, filed on May 29, 2020.

(51) Int. Cl. A61M 5/142; A61M 5/172

(52) U.S. Cl. CPC A61M 5/14276; A61M 5/172; A61M 2205/3569; A61M 2205/6054; A61M 2210/1003

(57) ABSTRACT

The present disclosure relates to an improved implantable drug delivery device, method, and system that is powered by compression and expansion of an inert gas contained within a compressible bellows and surrounded by a biocompatible housing that forms a reservoir which is connected to restricted channel for flow rate control and further connected to a flexible reinforced catheter that delivers medicants from the reservoir to the intrathecal space or other desired bodily tissue.

### PRIORITY

This application claims priority to U.S. Provisional Application 63/032,359 filed on May 29, 2021, which is incorporated by reference in its entirety.

### FIELD OF THE INVENTION

The present invention relates to an improved implantable intrathecal drug delivery device (IDDS), method, and system. More specifically, the present disclosure relates to devices, methods, and systems that are powered by a compressible, gas-filled or drug-filled bellows which allow for IDDS pumps of reduced size and weight with shortened catheter lengths.

### BACKGROUND OF THE INVENTION

Chronic pain is an insidious condition where patients with cancer, back injuries, and other conditions often require extraordinary treatments to resolve the condition. It is very common in severe cases that initial treatment delivery methods such as oral, intramuscular injection, or intravenous delivery methods lose effectiveness, and physicians switch to methods where drugs and other treatments are placed in or near the spinal space. Common treatments located in the spinal space for intractable chronic pain are electronic stimulation devices known as TENS units and implantable pumps for delivery of medications intrathecally. In intrathecal, or subarachnoid, drug delivery, pain medications and/or medications used for spasticity are introduced directly to the spinal fluid (intrathecal space) through an intrathecal drug delivery system (IDDS) comprised of an injection port, a reservoir, a low flow rate infusion pump, and a delivery catheter.

The intrathecal space is usually accessed from the lumbar region of the lower back. It is well known that to produce the same effectiveness or bioavailability of a drug delivered orally, subcutaneously, or intravenously requires a higher dosage than is required if the same analgesic agent is delivered intrathecally. Since intrathecal delivery requires less medication, many of the deleterious side effects are significantly reduced. In addition, there are certain medications that are only delivered intrathecally. One such medication is ziconotide, a non-opioid which can be effective at reducing pain, which must be delivered intrathecally due to its inability to cross the blood-brain barrier.

Within the drug field of intrathecal pain control, a wide variety of analgesic drugs are used. Since the rationale for the intrathecal delivery is a lower dosage for analgesic effect, the required volume for the same effect is greatly reduced compared to other forms of delivery, e.g. oral, intravenous, subcutaneous, transdermal etc. The reduction in volume allows for practitioners to utilize devices implanted in the body. As a result of the significant reduction in volume, the implantable pump design requires less frequent filling of the reservoir. Currently, the standard practice for refilling the reservoirs of implanted pumps is on a once-per-month basis. However, there are instances where the frequency of reservoir filling via injection ranges from once a week or once every several months.

The reduced volume and reduced dosage overcome many of the other undesirable side effects of analgesic drugs delivered by other routes, such as oral, intravenous, transdermal, and subcutaneous methods. For example, opioids are a common intrathecally-delivered analgesic agent. This drug class is well known to cause constipation due to opiate receptors in the gut and its effect on the motility of the intestinal tract. When this same drug class is delivered intrathecally, motility of the intestines remains intact. There are also many indications for the use of implantable drug delivery devices in the case of both malignant and non-malignant pain and spasticity.

In general, implantable drug delivery devices should be considered when other conservative therapies have failed and non-intrathecal therapy regimens (i.e., oral, subcutaneous, intramuscular, intravenous, or transdermal) are not adequately controlling symptoms and/or non-intrathecal regimens are causing significant side effects. Currently, the only medications that are approved by the U.S. Food and Drug Administration (FDA) for intrathecal administration are morphine, ziconotide, and baclofen. However, recent literature supports the use of many other opioid and non-opioid medications for patients who cannot tolerate the on-label FDA medications. The other medications must be obtained through special compounding pharmacies to ensure sterility and proper concentration. This is especially important when multiple medications are used in an implantable drug delivery system. Of note, there is ongoing research into novel non-opioid medications, such as resiniferatoxin, which has the potential to be helpful in treating cancer pain.

Typical intrathecal therapy for chronic pain control is to place a catheter into the subdural area, also described as the subarachnoid area, of the spine and into the intrathecal space. Infusion to the intrathecal space is achieved by connecting the spinal catheter to an implantable delivery device that is either a constant flow or programmable variable flow pump. Once connected, the clinician determines the appropriate dosage of medicant by titrating in a bolus delivery and monitoring the patient's level of analgesic effect. Once the adequate dosage and delivery is determined, the delivery system reservoir is filled via an injection through a self-sealing septum. In a fixed rate-type device, the pump action is controlled by compressing the gas chamber in the device when the reservoir is filled. Programmable pumps operate in a similar manner, but rates can be adjusted via transcutaneous communication with a microelectronic control system on board the implanted device. The programmable-type systems require battery power to operate, whereas the operation of the fixed rate devices needs no electrical current. The programmable devices thus require on-going review by clinicians to review and adjust the flow rate as necessary, as well as to monitor the remaining battery life. Upon depletion of battery life, a surgical procedure is required to replace the battery.

Regulations and recommendations by manufacturers require intrathecal drug delivery systems to be filled every 6 months, but the interval may vary. In general, IDDSs have been found to be very cost effective. In chronic non-cancer pain patients, IDDS is more effective than conventional medical management after around two years. However, in cancer pain patients, due to the dynamic nature of their pain, IDDSs can be cost effective in as little as three months.

Infusion of medications by means of implantable and subcutaneous constant rate pumps are taught by U.S. Pat. Nos. 3,731,681 5,731,681, 6,852,106, and 9,937,290. Disclosed are pump mechanisms with delivery catheters which utilize similar architecture and functionality. Although these implantable pumps are useful as configured, there are several problematic issues.

Most existing implantable pumps are somewhat heavy, bulky devices that require surgical insertion into a pocket on the front abdominal wall, and the catheter is tunneled subcutaneously and transversely across the flank of the abdomen wall to the base of the spine and into the intrathecal space. It is therefore desirable to have an implantable pump that is smaller in size to reduce bulk and weight. Another adverse event that may occur over time is the location of the pump can shift, which affects the location of the catheter tip and additionally risks dislocation or dislodgment of the catheter tip from the distal or proximal ends of the device. The reduction in weight and size would allow for smaller surgical sites and abdominal pockets, which would provide for a greater range of alternate locations for implantation of the device and would diminish the deleterious cosmetic effects of subcutaneous implantation of larger devices. Smaller implant devices can be surgically located in smaller tissue pockets and placed at anatomical sites that are undesirable for the traditional larger devices. A preferred location for implantation would be a site that is closer to spine area. This location would allow for surgical practitioners to reduce the length of the catheter tunnel, which has the benefit of reducing surgical tissue dissection and resultant trauma and surgery time. It also would reduce the volume of fluid contained in the catheter, often called “dead space.”

Thus, desirable features of an implanted pump include one of reduced size and weight with a shortened catheter length. Another desirable feature of an implanted pump is the ability to remotely interrogate the device for data and location information without transcutaneous or percutaneous punctures or access. In particular, the identification of the device, history of access, implantation date, and the amount of medication remaining in the reservoir are helpful parameters to ascertain without directly accessing the device.

### SUMMARY OF THE INVENTION

Disclosed herein is an apparatus design for an implanted intrathecal infusion pump and methods of using such a device. The implanted intrathecal infusion pump operates as a result of gas under pressure in a gas chamber exerting force on a reservoir containing a fluid, including for example a medicant and/or an infusate, contained in a sealed housing. The fluid is compressed by the gas as it collapses, which extrudes the fluid through capillary tubes into a catheter and further to the infusion site in the spinal intrathecal space. It is contained compression that receives the fluid to be stored. The design is further characterized by a reduced bulk of material that results in a minimalist, low profile, lightweight structure.

The primary purpose of the device is to provide a therapeutic agent to reduce pain. However, other uses of the device are also contemplated, including drug delivery applications to treat conditions such as spasticity, diabetes, cancer, etc.

Certain embodiments provide a flow control mechanism in the form of a microchannel panel, or capillary channel system. The microchannel panel receives a fluid input from the reservoir and outputs the fluid to the catheter to provide capillary flow control. The flow control panel can be formed by micromachining and/or resistive masking techniques to form the microchannels in a precise and repeatable manner.

Certain aspects allow a user to remotely interrogate the implanted pump via a handheld transponder that relays information from the implanted pump by querying data contained on an RFID chip embedded in the structure of the implanted pump. The transponder sends an identification (ID) code, and that the ID code is correct and data, latency and through-put, and be coordinated with the electromagnetic compatibility (EMC) performance of the implant, scanner and wireless data link.

In some aspects, a sensor system measures the reservoir volume status indirectly by using, for example, a two-part Hall effect sensor attached to the bottom-most portion of the bellows-type reservoir. The armature portion of the Hall effect sensor attaches to the reservoir, and the receiver reader channel of the sensor assembly is attached to the housing of the reservoir.

Some embodiments provide a palpation ring which can be palpated through the skin to locate the injectable septum for fluid injection. The ring and the reservoir housing chamber portion adjacent to the ring can be embedded with detection materials made of substances that are detectable with extracorporeal detection systems. The type of detection can include any detection modes that are routinely used in hospital settings, such as radiographic, ultrasonic, and magnetic imaging.

Also provided in some embodiments is a reinforced catheter and catheter attachment mechanism to withstand inadvertent puncture or detachment of the delivery catheter.

Polymer materials that do not interfere with MRI systems can be used for the reservoir housing and other components of the device. For example, a flexible elastomeric bladder may be used for the reservoir and may be constructed of a variety of suitable implantable materials, e.g., silicone, TPES, polyurethane, and/or PETG. In the case of the reservoir housing, a more rigid structural biocompatible type polymer may be used, e.g., PEEK, UHMWPE, polyamide, polysulfone, etc.

### BRIEF DESCRIPTION OF THE DRAWINGS

The accompanying drawings, which are incorporated in and constitute a part of this specification, illustrate various embodiments of the present disclosure, and, together with the description, serve to explain the principles of the disclosure. In the drawings:

FIG. 1 is a cutaway sectioned view of one example of an implantable intrathecal drug delivery apparatus with housing and elastomeric reservoir in a closed static full position.

FIG. 2 is an isometric top and side view of one example of an assembled implantable intrathecal drug delivery apparatus shown with a truncated catheter.

FIG. 3 is a schematic diagram of the electronic interrogation system used to remotely retrieve pump metrics and identification information.

FIG. 4. is a schematic diagram of an RFID system.

FIG. 5 is a front and side anatomical view of an implanted intrathecal drug delivery apparatus, a reinforced catheter, and a catheter attached to a port reservoir.

FIG. 6 is an illustration of a flow control capillary channel system and methods of manufacturing said flow control capillary channel system.

FIGS. 7A-D are illustrations of designs for the implantable drug delivery system.

FIG. 8 is a top and isometric view of one example of an implantable drug delivery system.

FIG. 9A is a crossection view of one example of implantable drug delivery system. FIG. 9B is a close up cross-section of one example of a refill mechanism.

FIGS. 10A-10D are illustrations of various embodiments of the refill mechanisms.

FIG. 11 illustrates examples of valving mechanisms that can be employed in implantable drug delivery systems.

FIG. 12 illustrates an example of a design that incorporates a textured bottom to enhance securement of the device.

FIG. 13 illustrates the operation of one example of a refill mechanism and refill needle design.

FIG. 14A illustrates valving mechanism that can be employed with an implantable drug delivery system to control the flow rate of the device.

FIG. 14B illustrates the changes in flow rate by rotation of the valving mechanism from low to no flow.

FIG. 15 illustrates an example of a catheter coupling design.

### DETAILED DESCRIPTION OF THE INVENTION

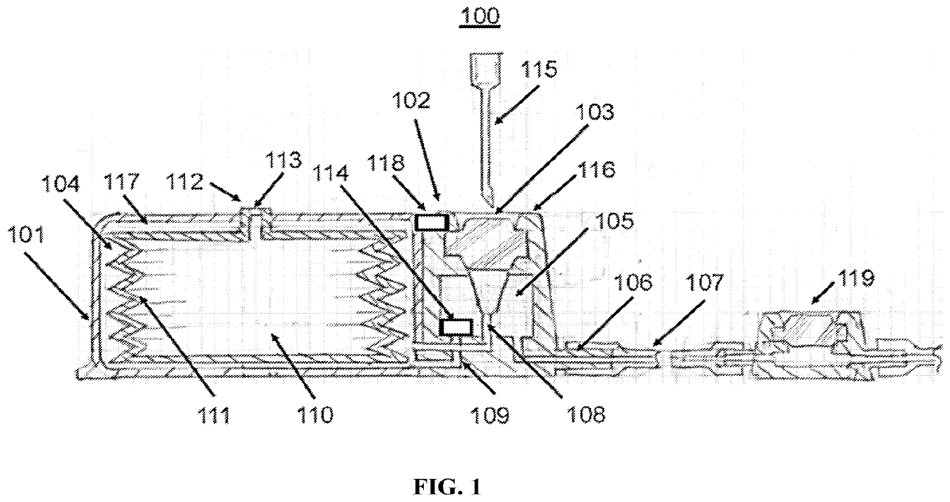

Referring now to FIGS. 1, 2, 3, 4, 5, 6, 7, 8, 9, 10, 11, 12, 13, 14, and/or 15 there is shown a variety perspective, schematic, illustrations, and section views of embodiments of the present invention.

Referring now to FIG. 1, shown is a section view of the implantable drug delivery system (IDDS) assembly 100 where housing 101 is a biocompatible apparatus which stores a fluid 117, which can be a medicant and/or an infusate, for example, inside a reservoir 104. The gas chamber 110 is an expandable bellows 111 that is located inside the reservoir 104 and contains an inert gas which is charged into the bellows 111 via the gas port 112 and permanently sealed by gas port plug 113. The fluid 117 is placed into the reservoir through the self-sealing, puncturable septum 103 by a non-coring injection needle 115 and upon injecting flows through the reservoir inflow channel 108 into the reservoir 104.

Surrounding the septum 103 is a raised rim 102 comprising a palpation ring which may be palpated using a touch sense to allow for locating and targeting the needle into the puncturable septum 103. The palpation ring can also include location sensors 116 and/or embedded materials that react to external extracorporeal readers for machine location of the septum.

The flow of the fluid 117 follows a path through the reservoir outflow channel 109, into a filter 114, to the microfabricated etched capillary channel system 105, and to external exit port tube 106 that is coupled to the proximal end of an exit catheter 107. The catheter may be a flexible reinforced catheter.

The IDDS assembly 100 may also include a separate in-line infusion port 119 for bolus injection into tissue via catheter conduit 107 that is tunneled to the intrathecal space or tissue to be infused.

Also included in some embodiments is an RFID chip 118 located inside the housing 101 for relaying embedded information and data on the IDDS status.

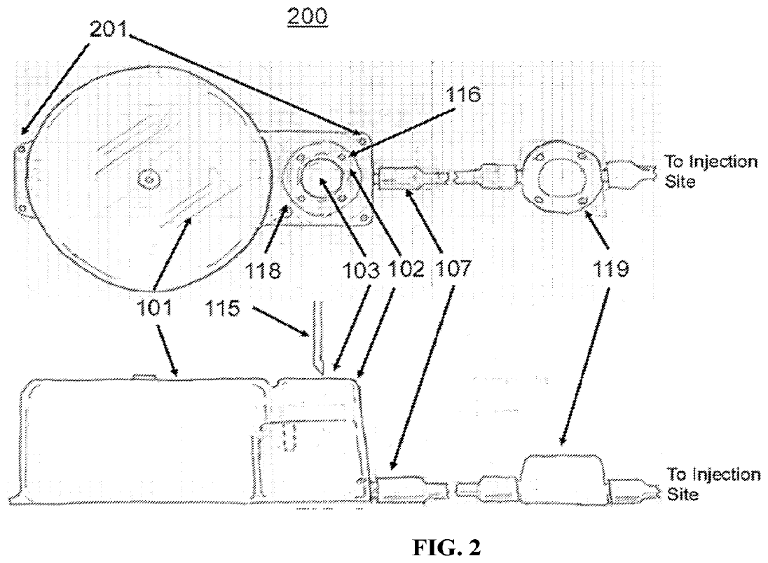

FIG. 2 provides isometric top and side views of the IDDS assembly 200. Key external components of the assembled IDDS including the housing 101, the puncturable septum 103, and adjacent palpation ring 102 are shown. The palpation ring may have location sensors 116 to guide location of the puncturable septum to deliver fluid into the IDDS. The fluid may be subsequently delivered to bodily tissues, such as the intrathecal space, via the exit catheter 107. Suture holes 201 at several locations on the housing 101 may provide attachment points for securement of the IDDS to tissues at a surgical site. Also shown in FIG. 2 is bolus port 119.

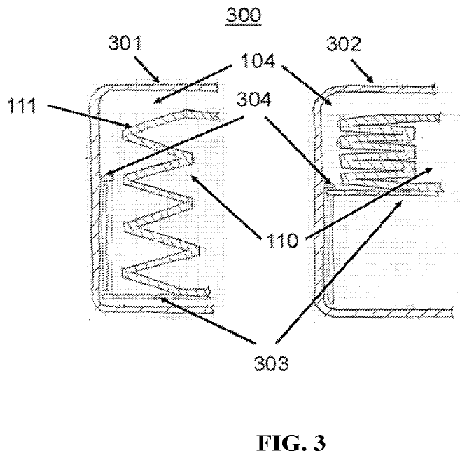

FIG. 3 provides a detailed cutaway view of an embodiment of a sensor assembly 300 which can be used to indirectly measure the volume status of fluid in the reservoir. Shown are two states 301 and 302 of the sensor, the sensor comprising a sealed expandable and collapsible bellows 111 comprising a gas chamber 110 containing inert gas. In 301, depicted is a full reservoir state in which the bellows 111 are in an expanded state and the gas chamber 110 is filled with gas. In 302, depicted is an empty reservoir state in which the bellows 111 are in a collapsed state with inert gas of the gas chamber 110 compressed. A bellows reservoir sensor arm 303 may be attached to the bottom of the bellows 111 and may be proximally aligned with linear positional sensor 304 that senses the location of the sensor arm 303 as it travels through a range of motion related to the state of the bellows 111. The state of the bellows 111 can be transmitted by the linear reader strip 304 to an external reader (not shown) to indicate changes in fluid volume, e.g., from a full reservoir state (expanded bellows 301 with gas chamber 110 filled with inert gas) to an empty reservoir state (collapsed bellows 302 with compressed inert gas in gas chamber 110).

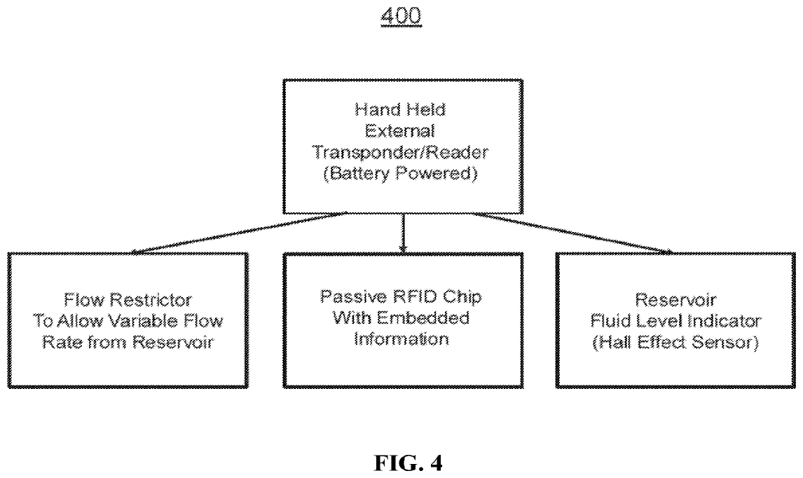

FIG. 4 is a schematic diagram 400 of a remote transponder and reader that can retrieve information from sensors within the implanted IDDS 100. The remote transponder/reader may be battery powered. Information to be retrieved may include information embedded on a passive RFID chip and/or the reservoir fluid level. The remote transponder/reader may also control a flow restrictor of the IDDS to provide for a variable flow rate from the reservoir into tissue.

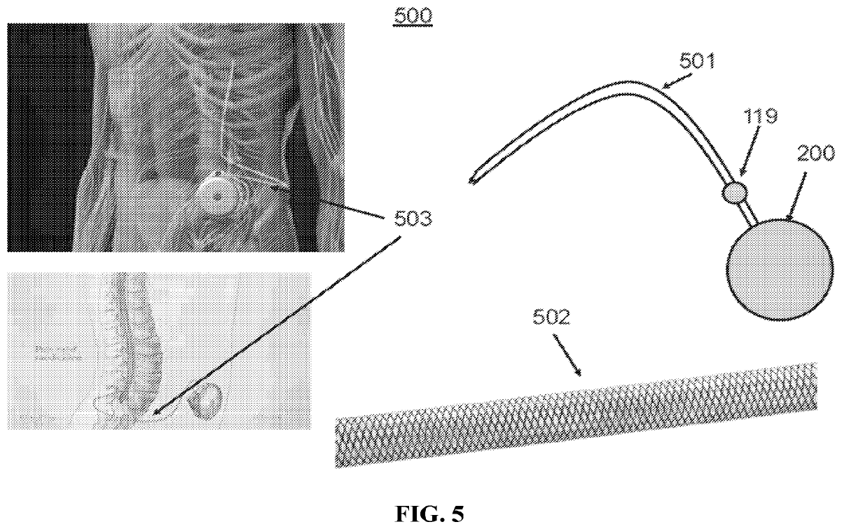

FIG. 5 is a series of illustrations 500 showing a reinforced catheter 502 which may be attached to the drug delivery device 200. The drug delivery device may be coupled via a catheter proximal section to a full-length catheter 501, the full-length catheter having a distal tip 503 located at the tissue site. As illustrated the IDDS can be attached to the abdominal wall, and the tunneled catheter 501 may be placed such that the distal tip 503 is located in the spine intrathecal space.

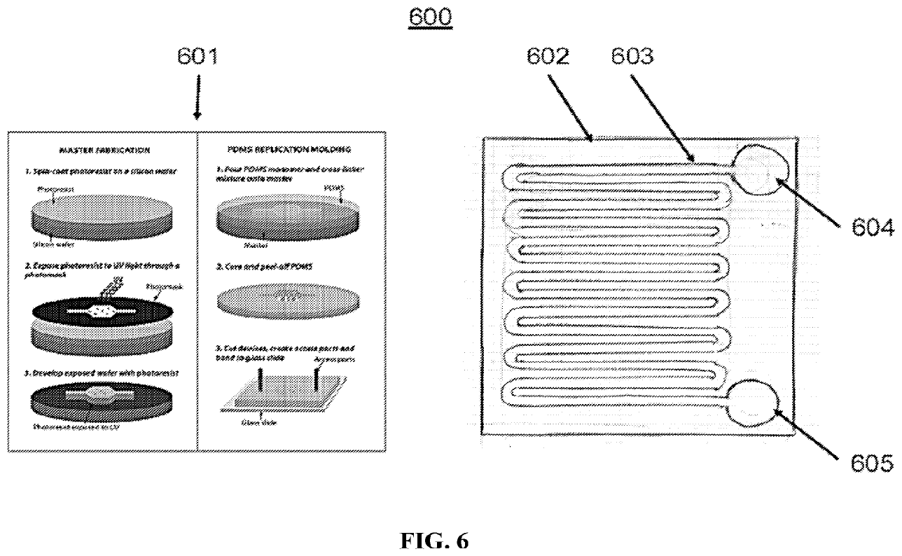

FIG. 6 illustrates a process by which a microfabricated etched capillary channel system may be manufactured 601 using, for example, MEMS or PDMS replication molding. In some embodiments, manufactured is a capillary bed configuration made of a membrane substrate 602 upon which a channel 603 is etched or formed, a capillary inlet port 604 coupled to the capillary channel system proximally, and a capillary outlet port 605 coupled to the capillary channel system distally.

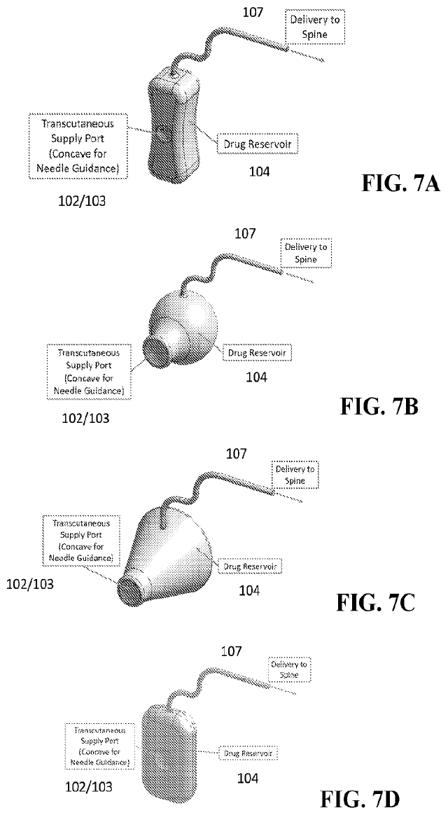

FIGS. 7A to 7D provide illustrations of designs for the implantable drug delivery system. The designs comprising a transcutaneous supply port 102/103, a drug reservoir 104, and a delivery conduit 107 that can be fluidly connected to a target site. The body of the device can be in various shapes and geometries and can have a concave portion for guiding a needle or other device for filling or refilling the reservoir.

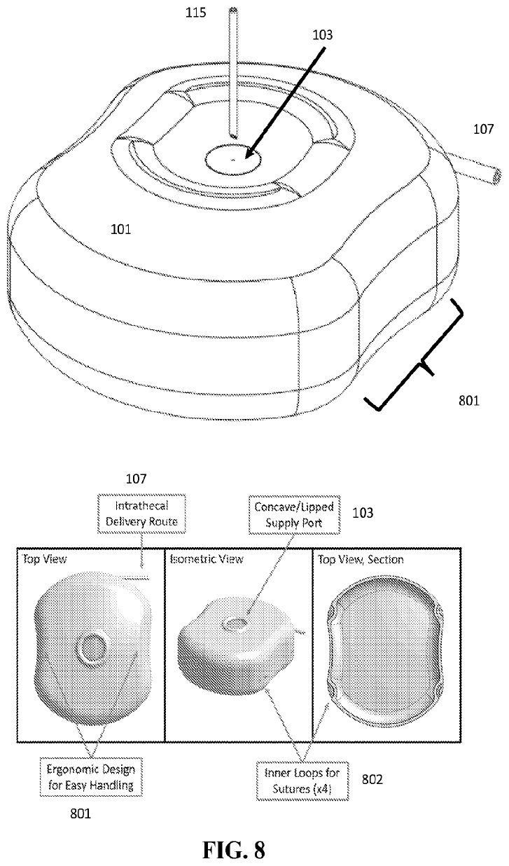

FIG. 8 is a top and isometric view of one example of an implantable drug delivery system. In this particular embodiment the housing (or body) 101 is configured easy handling and comprises a concave portion leading to the supply port 103, which can be punctured by needle 115. In certain aspects, any of the embodiments described herein can have the housing of the device configured to have an ergonomic design 801 to be easily graspable before and after implantation, the design including indentions or grips externally on one or more sides of the device housing to accommodate the fingers and/or thumb of persons handling the device. The device is configured with a catheter 107 providing an intrathecal delivery route. The implantable drug delivery system can have inner loops 802 for sutures.

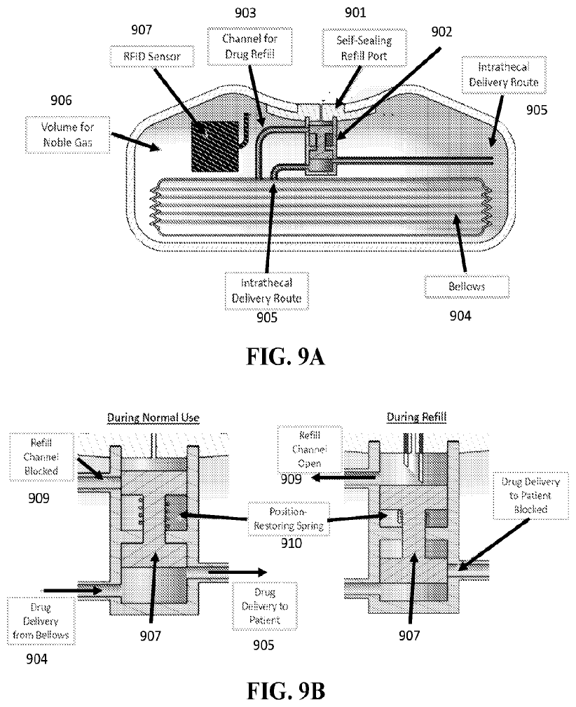

FIG. 9A is a cross-section view of one example of implantable drug delivery system. The concave portion of the body is shown providing access to refill port 901 and its valve mechanism 902 (see FIG. 9B for additional details). The valve mechanism 902 is fluidly connected to reservoir/bellows 903 through drug refill channel 903 and fluidly connects the reservoir 904 to the delivery component 905. In a first configuration solution to be delivered is contained in the volume of the body while a gas fills the bellows. In a second configuration the solution to be delivered is contained in the bellows and a gas fills the volume of the device body. Refill port 901 can include a puncturable septum. The body forms a cavity or body volume 906 that can be filled with a noble gas or similar gas. In certain aspects, the cavity 906 can contain a RFID sensor 907 and/or other electronics to monitor, modulate, or control operation of the device. The body cavity will also contain a bellows 904 that is the reservoir for a medicament to be delivered by the device.

FIG. 9B illustrates one example of valve mechanism that can be employed. In this aspect a two headed plunger 908 having a top portion and a bottom portion connected by a stem. When in use the top portion blocks fluid path 909 from the refill port 901 to the reservoir 904 and the bottom portion is positioned to allow flow from the reservoir 904 to the delivery component 905. The stem can be configured with a spring mechanism 910 that can be depressed during filling and restore the operational positioning of the plunger once filling is completed or ceased. In certain embodiments, the medicament to be delivered is stored in the reservoir of the housing, in other embodiments, the medicament can be stored in the cavity formed inside the bellows. Also, in the event the medicament is stored in the reservoir of the housing a gas can be introduced into the bellows, and alternatively, if a medicament is stored in the bellows a gas can be introduced into the reservoir of the housing.

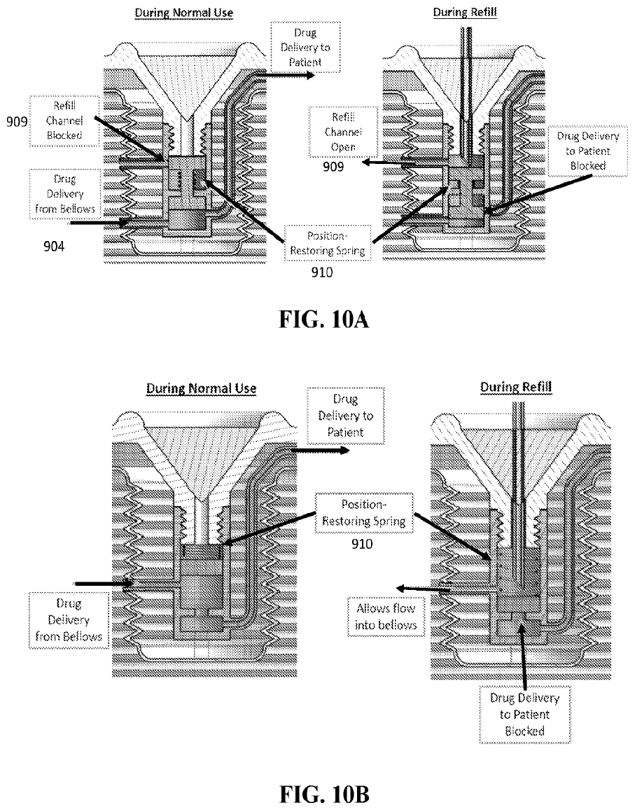

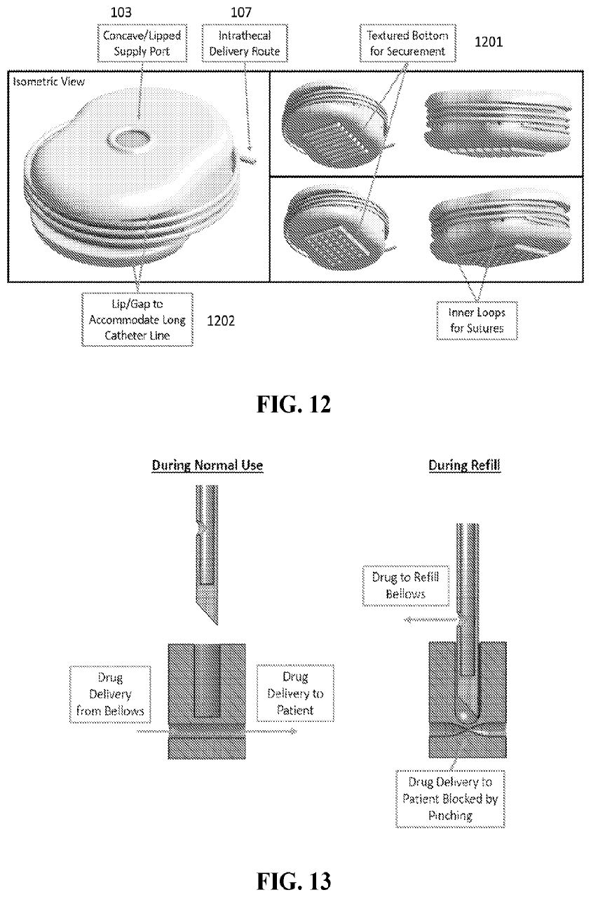

FIGS. 10A-10D are illustrations of various embodiments of the refill mechanisms. Various designs have been envisioned for valve designs for refill compatibility. The valve mechanisms allow for the drug delivery line to be temporarily blocked while the bellows or medicament volume is being refilled. This can be achieved by twisting a valve (FIG. 10C, 10D), compressing a delivery line (see FIG. 13), and/or incorporating a spring loaded valve (FIG. 10A, 10B). Refill compatibility can be improved with valve designs that do not produce a bolus of drug during refill. Certain aspects of the refill mechanism can include features to capture/trap the tip of refill needle during refill. FIG. 13 illustrates a refill mechanism and refill needle design that provides a pinch mechanism to modulate flow.

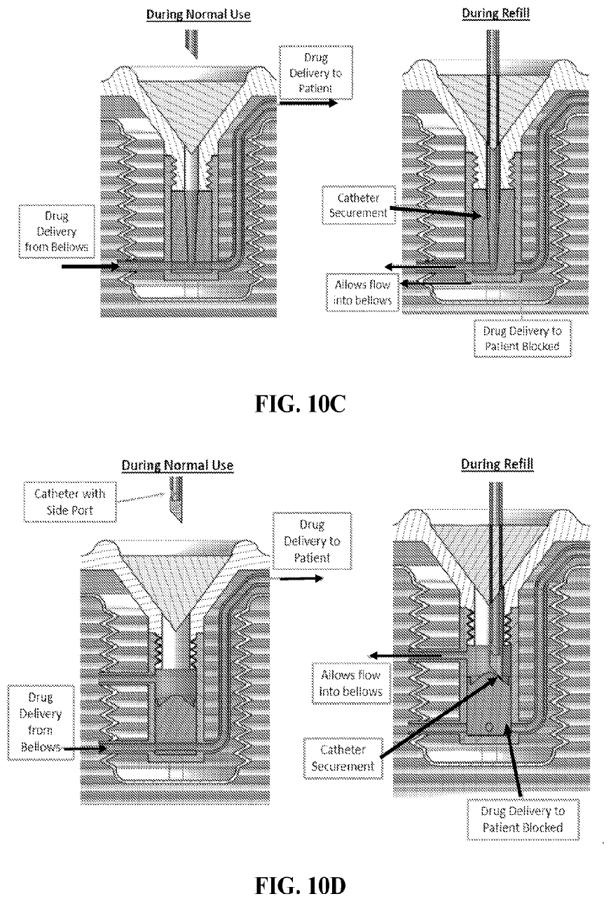

FIG. 11 illustrates examples of twisting valve mechanisms that can be employed in implantable drug delivery systems during use and during refill. In certain aspects, the refill channel is open to a needle or like instrument supplying a medicament while the delivery pathway is closed. When filling complete and the needle of like instrument is removed the refill channel can be closed and the delivery channel can then be opened. In certain aspects, the refill mechanism is spring loaded and returns to a delivery position after refilling is complete. In certain aspects, the channels can be opened and/or closed by turning or twisting a valve member, or vertical movement of a valve member, or constriction/relaxation of a catheter channel.

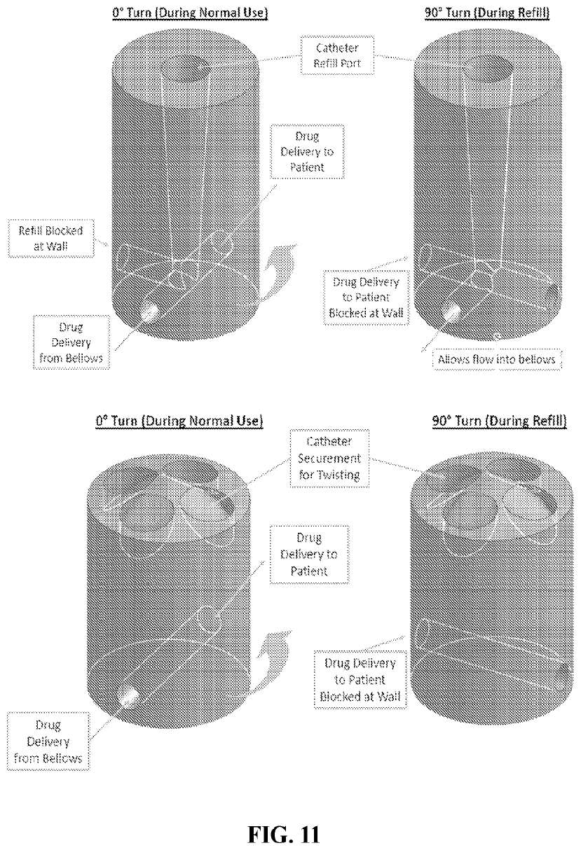

FIG. 12 illustrates an example of a design that incorporates a textured bottom 1201 to enhance securement of the device. The devices described herein can include a feature/lip 1202 to allow spooling of catheter line around device, this is illustrated in FIG. 12, and can be implement in the design of any device described herein. In addition the body can be configured with 1, 2, 3, 4, or more suture loops to aid in securing the device when deployed. The suture loop(s) can be position at various positions on the device, including, but not limited to the corners of the body. Another feature that can be included is the inclusion of one or more textured surface(s) on device to reduce movement of device (two variations (grid or rib variations) are shown in FIG. 13).

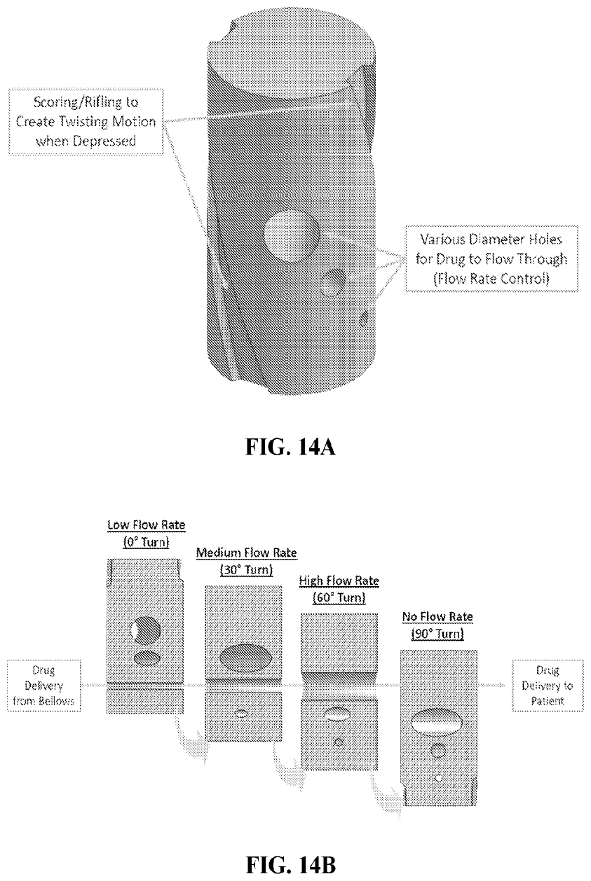

FIGS. 14A-14B illustrate a valving mechanism that can be employed with an implantable drug delivery system to control the flow rate of the device. Valve designs for various flow rates include an on/off (binary) valve, a valve with different diameter flow through holes, and/or a valve with scoring/rifling to induce twisting motion of the valve as it is depressed.

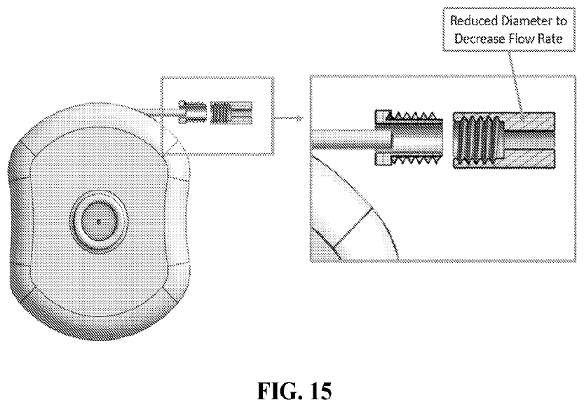

FIG. 15 illustrates an example of a catheter coupling design. In certain aspects, the IDDS can include a tunable flow control valve at end of catheter line, such as that illustrated in FIG. 15, where the diameter of a connection with the conduit to the target can be decreased to limit flow rate.

While the present disclosure has been set forth in terms of a specific embodiment or embodiments, it will be understood that the present apparatus and the method of using the same herein disclosed may be modified or altered by those skilled in the art to other configurations. Accordingly, the disclosure is to be broadly construed and limited only by the scope and spirit of the claims appended hereto.

The following table lists the reference characters of names or features used herein:

REFERENCE CHARACTER TABLE

| Number | Feature or Element |
|---|---|
| 100 | Intrathecal Drug Delivery System Section |
| 101 | Housing |
| 102 | Palpation Ring |
| 103 | Puncturable Septum |
| 104 | Reservoir |
| 105 | Microfabricated Etched Capillary Channel system |
| 106 | External Exit Port Tube |
| 107 | Exit Catheter |
| 108 | Reservoir Inflow Channel |
| 109 | Reservoir Outflow Channel |
| 110 | Gas Chamber |
| 111 | Bellows |
| 112 | Gas Injection Port |
| 113 | Gas Injection Port Seal |
| 114 | Reservoir Outlet Filter |
| 115 | Non-Coring Injection Needle |
| 116 | Location Sensors |
| 117 | Fluid |
| 118 | RFID Chip |
| 119 | Bolus Port |
| 200 | Exterior View of IDDS |
| 201 | Suture Holes |
| 300 | Sensor Assembly |
| 301 | Empty Reservoir State |
| 302 | Full Reservoir State |
| 303 | Bellows Reservoir Sensor Arm |
| 304 | Linear Positional Sensor |
| 400 | Diagram of Electronic Sensors & Reader |
| 500 | Catheter Placement & Reinforcement |
| 501 | Catheter |
| 502 | Reinforced Catheter Membrane |
| 503 | Anatomical Location of IDDS |
| 600 | Microfabricated Capillary Channels |
| 601 | Fabrication Methods |
| 602 | Membrane Substrate |
| 603 | Capillary Channel |
| 604 | Capillary Inlet Port |
| 605 | Capillary Outlet Port |
| 801 | Ergonomic inset |
| 802 | Suture loops |
| 901 | Self Sealing Refill Port |
| 902 | Valve mechanism |
| 903 | Refill channel |
| 904 | Bellows |
| 905 | Delivery route |
| 906 | Device volume |
| 907 | Valve piston |
| 909 | Drug delivery from reservoir |
| 910 | Piston spring |
| 1201 | Textured bottom |
| 1202 | Lip for catheter wrap |

1. An implantable intrathecal drug delivery device comprising:
a housing;
a sensor assembly comprising:
a compressible bellows enclosed by the housing, the bellows comprising a gas chamber filled with an inert gas; and
a bellows sensor arm coupled to the bottom of the bellows and proximally aligned with a linear positional sensor; and
a reservoir formed by an inner wall of the housing and an outer wall of the bellows;
wherein the reservoir contains a fluid, and wherein the reservoir is in fluid connection with a catheter for delivery of the fluid to a terminal site.

2. The device of claim 1, wherein the device is configured to deliver fluids to a terminal site at a fixed flow rate.

3. The device of claim 1, further comprising a microfabricated capillary channel in fluid connection with the reservoir for restrictive flow of the fluid to the catheter.

4. The device of claim 1, where the reservoir comprises a bladder, and wherein the bladder is made of elastomeric material.

5. The device of claim 1, wherein the sensor assembly is configured to detect the volume of fluid in the reservoir by detecting an expanded or compressed state of the bellows, wherein the reservoir is empty when the bellows are fully expanded or the reservoir is full when the bellows are fully compressed.

6. (canceled)

7. (canceled)

8. The device of claim 1, comprising a data information storage medium, where the data information can be retrieved transcutaneously from the device using an external transponder.

9. The device of claim 8, comprising an embedded RFID chip data information related to IDDS status.

10. The device of claim 8, wherein the external transponder is configured to retrieve data related to the volume of fluid contained within the reservoir from the sensor assembly.

11. The device of claim 1, further comprising a puncturable septum in fluid communication with the reservoir.

12. The device of claim 11, wherein the puncturable septum is surrounded by a raised rim capable of being palpated to locate the puncturable septum.

13. (canceled)

14. (canceled)

15. An implantable intrathecal drug delivery device comprising:
a housing;
a sensor assembly comprising:
a compressible bellows enclosed by the housing, the bellows forming a reservoir for a fluid to be delivered by the device; and
a bellows reservoir sensor arm coupled to the bottom of the bellows and proximally aligned with a linear positional sensor; and
a chamber formed by an inner wall of the housing and an outer wall of the bellows filled with an inert gas;
wherein the reservoir contains a fluid, and wherein the reservoir is in fluid connection with a catheter for delivery of the fluid to a terminal site.

16. The device of claim 15, wherein the device is configured to deliver fluids to a terminal site at a fixed flow rate.

17. The device of claim 15, further comprising a microfabricated capillary channel in fluid connection with the reservoir for restrictive flow of the fluid to the catheter.

18. The device of claim 15, where the chamber comprises a bladder, and wherein the bladder is made of elastomeric material.

19. The device of claim 15, wherein the sensor assembly is configured to detect the volume of fluid in the reservoir by detecting a compression state of the bellows, wherein the reservoir is full when the bellows are fully expanded, or the reservoir is empty when the bellows are fully compressed.

20. (canceled)

21. (canceled)

22. The device of claim 15, further comprising a data information storage medium, where the data information can be retrieved transcutaneously from the device using an external transponder.

23. The device of claim 22, wherein the device further comprises an RFID chip containing data information related to IDDS status.

24. (canceled)

25. The device of claim 15, further comprising a puncturable septum in fluid communication with the reservoir.

26. The device of claim 25, wherein the puncturable septum is surrounded by a raised rim capable of being palpated to locate the puncturable septum.

27. (canceled)

28. (canceled)
